(19) United States
(12) Patent Application Publication
(10) Pub. No.: US 2012/0259230 A1
(43) Pub. Date: Oct. 11, 2012

(54) TOOL FOR RECORDING PATIENT WOUND HISTORY

(76) Inventor: Elven Riley, Madison, NJ (US)

(21) Appl. No.: 13/430,805

(22) Filed: Mar. 27, 2012

Related U.S. Application Data
(60) Provisional application No. 61/474,139, filed on Apr. 11, 2011.

(51) Int. Cl. A61B 6/00

(52) U.S. Cl. 600/477

(57) ABSTRACT

An apparatus for reliably recording, and reproducing, a history of a patient's wound using the cameras readily available on smart phone or blackberries by taking a digital record image of the wound along with a fiducial marker that has both shape and color fiducial elements. The image then contains sufficient information so that any display may be adjusted to accurately display the original color, and to accurately deduce the original size and shape of the wound. By transmitting the recorded images to a central database having image analysis capability, real time information may be made available indicating any significant changes that have occurred since the last recording of the same wound, providing an opportunity for the attending physician to give further attention to the patient without a time consuming second visit.

### CLAIM OF PRIORITY

This application claims priority from U.S. provisional application 61/474,139 filed on Apr. 11, 2011.

### FIELD OF THE INVENTION

The invention relates to a system, apparatus and method for measuring the size and color of wounds, and more particularly to providing fiducials and ranging mechanisms to facilitate the use of smart phone digital cameras for recording digital color images of wounds for reference and measurement.

### BACKGROUND OF THE INVENTION

The existing standard practice for measuring wounds is for a trained nurse to trace the perimeter of the wound onto clear plastic film and then transfer the tracing to graph paper. It is widely used because it is easy to perform; it requires no special expertise or equipment and is therefore a low cost option.

The method does, however, have drawbacks. It is inconvenient, subjective and time-consuming and may also be painful for the patient. It is also a potential source of cross-contamination.

Although low cost digital cameras are now included in almost all cell phones, blackberries and smart phones, their use in measuring wounds has been limited by two major concerns: the uncertainty of the magnification of the digital image, and inaccurate reproduction of the color of the image.

The present invention solves these problems by providing a fiducial marker that has both color and shape reference information. A digital image that includes a view of the fiducial marker of this invention, when it is located near to, and in the same plane as the wound, allows any display of that image to be adjusted to be accurate to the original scene in color and in size.

The present invention may also include a simple, optical range finder to facilitate accurate and optimal positioning and orientation of the digital camera with respect to the wound.

### DESCRIPTION OF THE RELATED ART

The relevant prior art involving wound measurement includes:

U.S. Pat. No. 7,401,413 issued to Nelson on Jul. 22, 2008 entitled “Disposable wound measuring device” that describes a disposable wound measuring device for making simultaneous three-dimensional (length, width and depth) measurements of a wound or ulcer to facilitate wound care assessment and healing that includes a flexible, clear plastic planar card or member superposed on the wound and having a centrally located aperture and concentric circles radially arranged about the aperture, and an elongated depth gauge insertable through the aperture and incrementally adjustable either vertically or obliquely through the aperture and relative to the wound surface and the plastic member for making a wound depth measurement with the depth gauge being retained in position by a plurality of beads equidistantly spaced along the gauge and slightly larger in diameter than the aperture thereby self-retaining the depth gauge in a particular position for making the wound depth measurement.

U.S. Design Pat. No. D634,009 issued to Lam , et al. on Mar. 8, 2011 entitled “Color scale for providing consultation of skin condition” that describes an ornamental design for a color scale for providing consultation of skin condition, as shown and described.

U.S. Pat. No. 7,662,112 issued to Zamierowski , et al. on Feb. 16, 2010 entitled “Medical measuring device and method” that describes a device and method for measuring a subsurface wound or cavity of a patient. The device includes a flexible strip having first and second sections, the sections having first and second ends respectively with an intermediate bend being located between the first and second ends. The first end is adapted for being located internally to a patient, the second end being adapted for being located externally to the patient. The first and second sections have generally concave-shaped cross-sections with measuring indicia being located along at least one of the sections. The device also optionally includes a measuring strip and a receiver mounted on the second end for receiving the measuring strip, where the measuring strip and the flexible strip form a complementary structure for measuring a subsurface wound or cavity.

Various implements are known in the art, but fail to address all of the problems solved by the invention described herein. One embodiment of this invention is illustrated in the accompanying drawings and will be described in more detail herein below.

### SUMMARY OF THE INVENTION

The present invention relates to a system, apparatus and method for recording patient wound history.

In a preferred embodiment, an image recording device such as, but not limited to, a cell phone, a blackberry or a smart phone camera, may be used to record a digital record image of the patient's wound along with a fiducial marker that has both shape and color elements.

In this way, the digital record image may contain sufficient information that the original color and size of the wound may be determined at any later time, and that any display of the image may be adjusted to reproduce both the original color and shape, or size, of the patient's wound.

The fiducial marker may have both a shape element and a color element. The fiducial marker is preferably placed in proximity to, and preferably in the same plane as, the patient's wound when the digital record image is taken.

The image recording device may also have a range establishing element that may indicate when the device is located at, or close to, a fixed, predetermined distance from the patient's wound. This fixed, predetermined distance may, for instance, be an optimal distance for recording such images.

In a preferred embodiment, there may also be a patient identification element and a time of image element that establishes a time, a date and a patient identity associated with said digital record image obtained by said image recording device. This may, for instance, take the form of a bar code, or other encrypted or partially encrypted data image that may be digitally captured as a part of the digital record image.

In a preferred embodiment, the image recording device may also include a file compositing device that may combine the digital record image, the patient identity and the time and said date of acquisition data in at least one computer readable file.

The image recording device may include a range establishing element. This may, for instance, take the form of two or more light beams emitted by an element such as, but not limited to, a light emitting diode (LED) or a semiconductor laser or some combination thereof.

The recording device may also include appropriate circuitry for storing least one computer readable file, and appropriate circuitry for transmitting the file to another device such as, but not limited to, to a central data base. The central data base may be accessible by other users or computers such as, but not limited to, medical practitioner workstations.

Therefore, the present invention succeeds in conferring the following, and others not mentioned, desirable and useful benefits and objectives.

It is an object of the present invention to provide a blackberry type device with suitable augmentation to aid in fixing to record a high resolution, color image of a patient's wound.

It is another object of the present invention to provide images of wounds that may be rapidly uploaded to a central data base.

Yet another object of the present invention is to provide location information with the image.

Still another object of the present invention is to provide a server capable of calculating size and shape changes of a patient's wound with respect to previous images.

Still another object of the present invention is to provide the calculated size and shape changes in real-time so that any significant changes may be attended to while the physicians are already on site.

Yet another object of the present invention is to provide a wound recording system that can record all required visit information within 10 minutes.

### BRIEF DESCRIPTION OF THE DRAWINGS

FIG. 1 shows a schematic view of the system of the present invention.

FIG. 2 shows an image of a wound being taken using the apparatus of the present invention.

FIG. 3 shows an image of a wound taken using the apparatus of the present invention.

FIG. 4 shows side view of the apparatus of the present invention in use.

FIG. 5 shows a mobile camera device enhanced to function as a recording device according to the present invention.

FIG. 6 shows an isometric view of a fiducial marker of the present invention.

FIG. 7 shows a plan view of a fiducial marker of the present invention.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

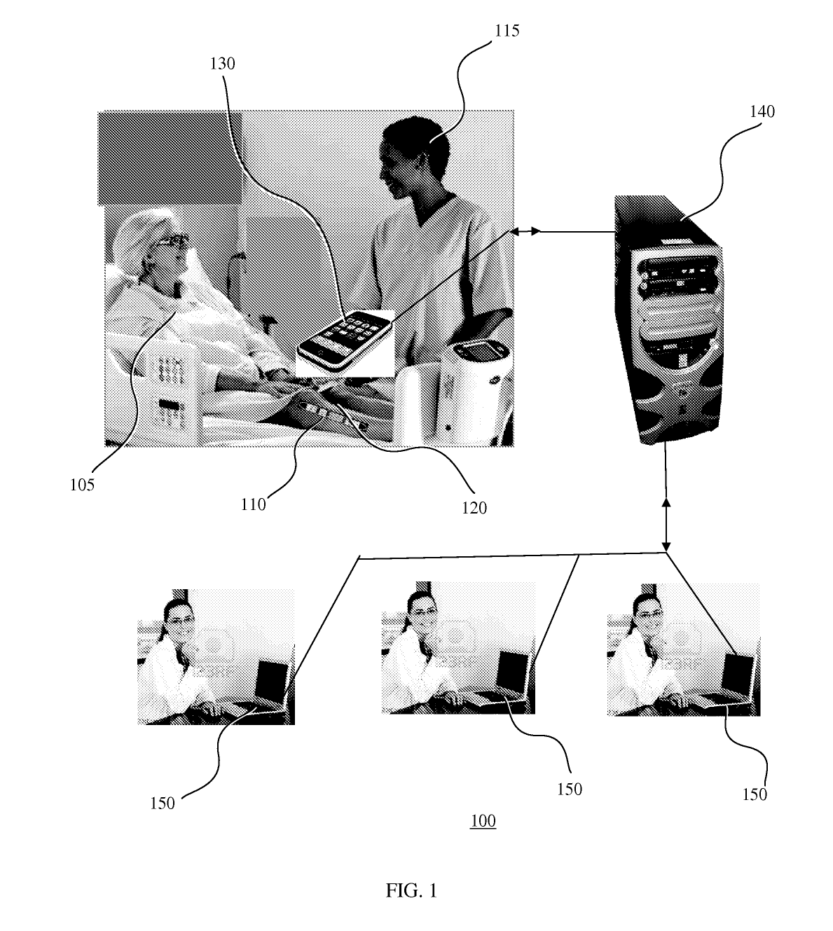

FIG. 1 shows a schematic view of a system for recording patient wound history 100 of the present invention.

The data gathering portion of recording a patient wound history 100 may occur at a patient's bedside. A suitably trained medical practitioner 115 may obtain a digital record image 250 using a suitable image recording device 130. The image recording device 130 may be used to take an image of patient's wound 120 that may include a fiducial marker 110. In a preferred embodiment, the fiducial marker 110 may have both a shape element 210 and a color element 220. The shape element 210 may be used to correctly size the wound even when displayed at an unknown resolution or magnification. The color element 220 may be used to ensure that the patient's wound 120 is displayed with correct color, and preferably with a same contrast of the original setting.

Once the digital record image 250 has been obtained, it may be transmitted to one or more central data bases 140 by any suitable data connection means such as, but not limited to, a wireless telephone, the internet, a landline or a fiber optic cable or some combination thereof.

The digital record image 250 may be accessible to authorized care givers on a device such as, but not limited to, a medical practitioner's workstation 150, a mobile phone, a note pad, a smart phone or some combination thereof.

In a preferred embodiment, the central data base 140 may include software or hardware that may analysis the digital record image 250 in real time by, for instance, comparing the wound size, shape or color to the same attributes in a previous image of the same patient's wound 120. That analysis may be available to the trained medical practitioner 115 while they are still by the patient 105, so that if any significant changes are seen special attention may be given to the patient 105 while the trained medical practitioner 115 is already on site, saving additional visits to the patient.

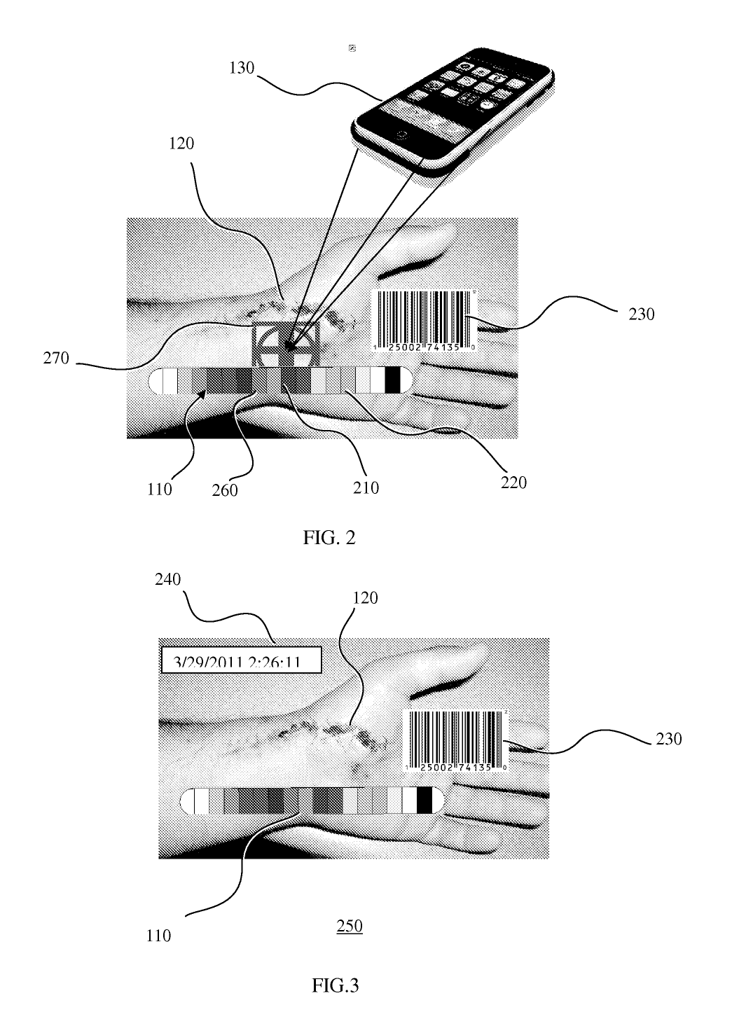

FIG. 2 shows an image of a wound being taken using the apparatus of the present invention.

A fiducial marker 110 having a plurality of fiducial marker segments 260 may be placed in proximity to the patient's wound 120 without obscuring the wound. The fiducial marker segments 260 may constitute both the shape element 210 and the color element 220 of the fiducial marker 110. A preferred placement of the fiducial marker 110 is in the same plane as the wound.

A patient identification element 230 may also be placed in close enough proximity to be included in the digital record image 250 without obscuring the patient's wound 120. The patient identification element 230 may, for instance, be suitably encrypted in an optical machine readable form such as, but not limited to, a one or two dimensional bar-code. The information itself may be further encrypted to provide appropriate patient privacy.

The image recording device 130 may then be positioned at a fixed, predetermined distance 330 from the patient's wound 120 by means of a ranging attachment to the digital record image 250. The ranging attachment may, for instance, be a triangulating device such as, but not limited to, two or more beams of light projected down from the underside of the image recording device 130 at an appropriate angle such that when the beams overlap on a surface, the projecting device is a fixed, predetermined distance 330 from the surface they are overlapping on.

In a preferred embodiment, the triangulation device may have three beams projectors, located along three different edges of the image recording device 130 housing, each of which projects an image of a different range establishing mark 270. The range establishing mark 270 may, for instance, be a symbol such as, but not limited to, a cross, a circle, a square or some other suitable geometric shape.

In a preferred embodiment, when the three range establishing marks 270 overlap on a surface in the plane of the patient's wound 120 and the fiducial marker 110, the camera may be positioned in close proximity to a fixed, predetermined distance 330 from that surface, and in a plane parallel to that surface. For the cameras used in most current mobile devices and smart phones, the fixed, predetermined distance 330 is preferably in a range of 5 to 15 inches, and in a more preferred embodiment in a range of 9 to 11 inches.

FIG. 3 shows digital record image 250 of a wound taken using the apparatus of the present invention.

The digital record image 250 may be a full color, digital image that shows the patient's wound 120 in part, or in its entirety, along with a fiducial marker 110. There is preferably also patient identification element 230 in the digital record image 250. The patient identification element 230 may include information such as, but not limited to, patient name, date of birth, location of the patient, type of medical insurance, or some combination thereof.

The digital record image 250 also preferably includes a time of image element 240 that may, for instance, be added by the image recording device 130.

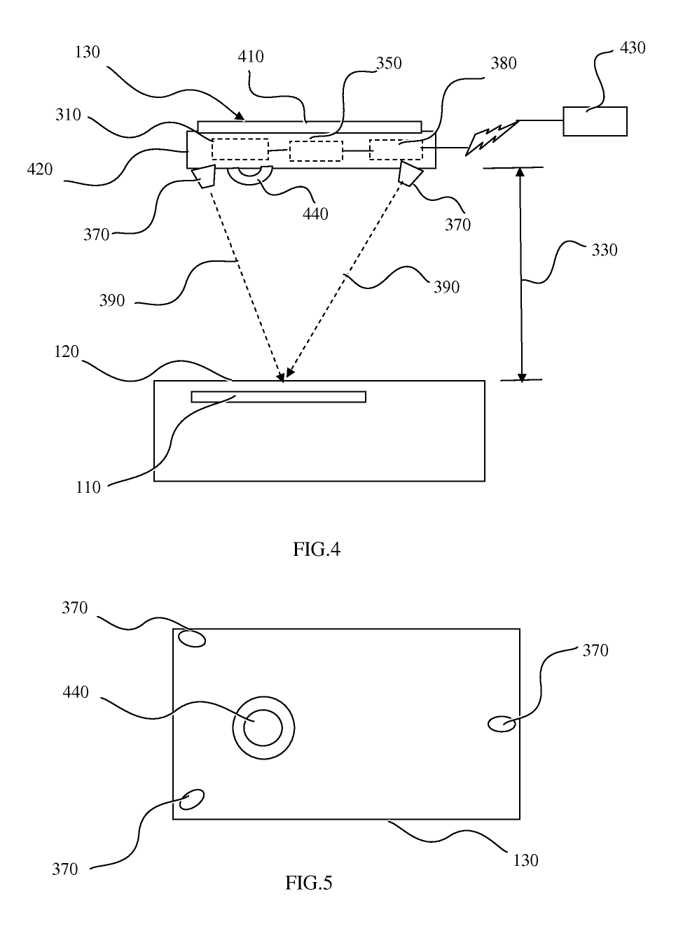

FIG. 4 shows a side view of the apparatus of the present invention in use.

The image recording device 130 may, for instance, be a smart phone equipped with a camera and enhanced with range finder for accurate positioning and orientation. The range finder may, for instance, include an attachment apparatus such as, but not limited to, a device skin 420 that holds one or more beam projectors 370. These beam projectors 370 may, for instance, be light emitting diodes and the may be fitted to the device skin 420 so that the optical beams 390 they emit are angled in. When the angled in optical beams 390 converge on a surface, the camera is a fixed, predetermined distance 330 from the surface it converges on.

Such a range finding device may be enhanced by having at least three beam projectors 370, configured so that the three beams converge to a single point when the image recording device 130, or preferably, the camera optical imaging element 440, is located a fixed, predetermined distance 330 from the surface it is converging on. The surface the beams converge on is preferably the plane containing the patient's wound 120 and the fiducial marker 110.

The image recording device 130 may include a storage element 310, a file compositing device 350 and a data transmission element 380. These may be linked together and may be parts or code modules operating on a smart phone digital processor and its related components.

The storage element 310 may store one or more digital record images 250. The file compositing device 350 may combine the digital record images 250 with further information such as, but not limited to, time, date, location, facility, equipment operator, the type of equipment being used, or some combination thereof. This information may be added to the digital record image 250 as an integral part of the image, or attached to the image as, for instance, a file header.

The data transmission element 380 may transmit one or more digital record image 250 directly back to the central data base 140, or it may transmit the data to a local relay 430 that may transmit the data on to the central data base 140.

FIG. 5 shows a mobile camera device enhanced to function as a recording device according to the present invention, seen from the bottom, or lens side of the image recording device 130. This view of the image recording device 130 shows the camera optical imaging element 440 and the beam projectors 370. The camera optical imaging element 440 may be a suitable focusing lens, a suitable focusing mirror, or some combination or variation thereof. The beam projector 370 may, for instance, also be simple pen light on/off devices such as, but not limited to, diode lasers. They may simply project a beam, or they may project an image or symbol such as, but not limited to, a plus sign, a box, a circle or some combination or variation thereof.

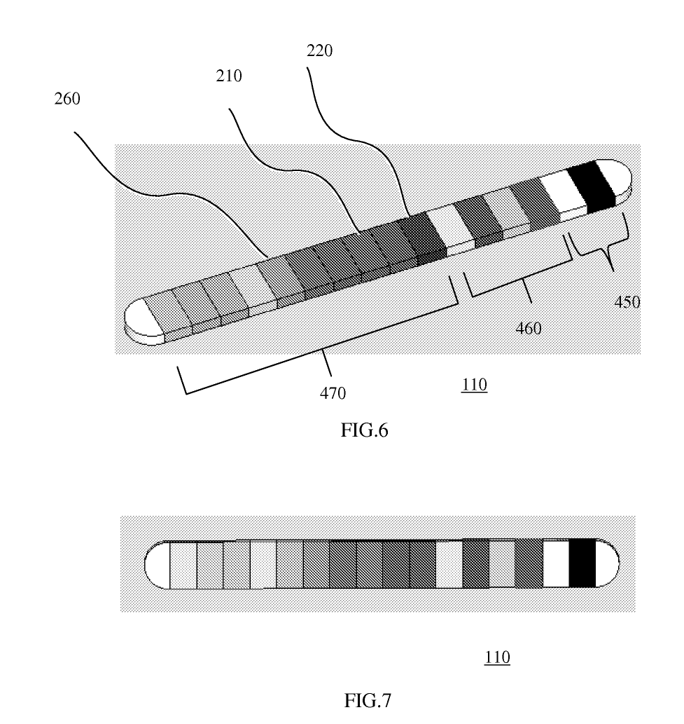

FIG. 6 shows an isometric view of a fiducial marker of the present invention.

The fiducial marker 110 may be divided into a plurality of fiducial marker segments 260. In a preferred embodiment, the fiducial marker segments 260 may all be equal in length and all equal in width. The square or rectangular fiducial marker segments 260 may then provide a shape element 210 that is a verification of the actual size of the wound, independent of any calculations regarding the magnification of the image.

To facilitate measuring the size of wound, the widths and lengths of the fiducial marker segments 260 may be multiples or simple fractions of an inch or a centimeter. Similarly, the widths and lengths of the fiducial marker segments 260 may be multiples or decimal fractions of an inch or a centimeter.

The fiducial marker segments 260 may be colored different colors so as to also provide a color element 220 to the fiducial marker 110.

The fiducial marker 110 may have a display color fiducial region 460 that may comprise at least one fiducial marker segment 260 colored in one of the three primary colors used in emission displays, namely red, green and blue. For example, the color fiducial region 460 may include at least three contiguous fiducial marker segments that are rectangular regions, one colored in a saturated red color, one colored in a saturated green color, and one colored in a saturated blue color. The display color fiducial region 460 may also, or instead have at least one of the fiducial marker segments 260 colored in the primary colors used to print reflective color images, namely cyan, magenta and yellow.

A purpose of these primary colors may be to provide clear references for adjusting the color balance on a display being used to show the digital record image 250 so that the wound colors are true to how they appeared on the patient.

The fiducial marker 110 may have a contrast fiducial region 450 that may, for instance, be fiducial marker segments 260 colored in black or white. The colors in this region may be used to adjust the contrast on a display being used to show the digital record image 250 so that the wound colors are true to how they appeared on the patient.

The fiducial marker 110 may also have a wound color fiducial region 470 in which the fiducial marker segments 260 are colored in a variety of colors often found in wounds, particularly colors that may be inductive of either positive healing or of problems in the wound healing. The wound color fiducial region 470 may include at least 5 contiguous fiducial marker segments that are rectangular regions, each colored in a color typically found in a human flesh wound. This color region may serve as a region of reference.

FIG. 7 shows a plan view of a fiducial marker of the present invention.

The body of the fiducial marker 110 may be made of a suitable substance that prints true such as, but not limited to, a wood or plastic stick, printed paper or cardboard, printed paper with post-it-note glue for temporary attachment, a wood or plastic stick with a tacky adhesive for temporary sticking to a surface, or some combination thereof.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed:

1. A fiducial marker optimized as a reference for recording a patient's wound history, comprising:
a plurality of fiducial marker segments each having a shape element and a color element, said fiducial marker segments arranged as a contiguous linear surface and comprising:
a display color fiducial region; and
a wound color fiducial region.

2. The fiducial marker of claim 1, wherein said display color fiducial region comprises at least three contiguous fiducial marker segments that are rectangular regions, one colored in a saturated red color, one colored in a saturated green color, and one colored in a saturated blue color; and wherein said wound color fiducial region comprises at least 5 contiguous fiducial marker segments that are rectangular regions, each colored in a color typically found in a human flesh wound.

3. The fiducial marker of claim 3, further comprising a contrast fiducial region comprising at least two contiguous fiducial marker segments that are rectangular regions, one colored in a saturated black color, one colored in a saturated white color.

4. The fiducial marker of claim 1 wherein said fiducial marker is one of a stick, a printed paper or a printed paper with post-it-note glue for temporary attachment.

5. The fiducial marker of claim 1 wherein said fiducial marker has at least four contiguous fiducial marker segments that are rectangles of an equal, known width and length and wherein each of the four contiguous fiducial marker segments are colored in a different color.

6. The fiducial marker of claim 5, where said known widths and lengths are multiples or simple fractions of an inch.

7. The fiducial marker of claim 5, where said known widths and lengths are multiples or decimal fractions of an inch.

8. The fiducial marker of claim 5, where said known widths and lengths are multiples or decimal fractions of a centimeter.

9. A system for recording a patient's wound history, comprising:
a fiducial marker having a shape element and a color element, said fiducial marker being located in proximity to said patient's wound;
an image recording device having a storage element; and
a range establishing element that indicates when said image recording device is located in close proximity to a fixed, predetermined distance from said patient's wound and said fiducial marker.

10. The system of claim 9 wherein said image recording device is a smart phone camera incorporated in a smart phone, and said range establishing element comprises at least two beam projectors rigidly attached to a device skin that is sized and shape to securely fit said smart phone, and wherein said beam projectors are aligned so as to emit optical beams that overlap at said fixed, predetermined distance.

11. The system of claim 10 further comprising a third beam projectors rigidly attached to said device skin and aligned so as to emit an optical beam that overlaps with said other optical beams when said smart phone camera is at said fixed, predetermined distance and the optical axis of said smart phone camera is oriented substantially normal to a plane containing said patient wound and said fiducial marker.

12. The system of claim 9 further comprising a patient identification element and a time of image element that establish a time, a date and a patient identity associated with said digital record image obtained by said image recording device; and
a file compositing device that combines said digital record image and said patient's identity and said time and said date of acquisition information into at least one computer readable file.

13. the system of claim 9 wherein said image recording device further comprises an element for transmitting said at least one computer readable file to a central data base.

14. the system of claim 13 further comprising a network of medical practitioner workstations linked to said central data base.

15. A method for recording patient wound history, comprising:
locating a fiducial marker having a plurality of shape elements and color elements, in proximity to said patient wound;
positioning an image recording device in close proximity to a fixed, predetermined distance from said patient's wound and said fiducial marker; and
recording an digital record image of both said patient sore and of said shape and color elements of said fiducial marker from said fixed, predetermined distance.

16. The method of claim 15 said image recording device is a smart phone camera incorporated in a smart phone, and said range establishing element comprises at least two beam projectors rigidly attached to a device skin that is sized and shape to securely fit said smart phone, and wherein said beam projectors are aligned so as to emit optical beams that overlap at said fixed, predetermined distance.

17. The method of claim 16 wherein said range establishing element further comprises a third beam projectors rigidly attached to said device skin and aligned so as to emit an optical beam that overlaps with said other optical beams when said smart phone camera is at said fixed, predetermined distance and the optical axis of said smart phone camera is oriented substantially normal to a plane containing said patient wound and said fiducial marker.

18. The method of claim 15 further comprising transmitting said at least one computer readable file to a central data base.
